(19) United States
(12) Patent Application Publication
(10) Pub. No.: US 2016/0346179 A1
(43) Pub. Date: Dec. 1, 2016

(54) Dual Component Oral Composition for Enhancing remineralization of teeth

(71) Applicants: Kimberlee U. Martin, Houston, TX (US); Thomas Ray O'Brien, Houston, TX (US)

(72) Inventors: Kimberlee U. Martin, Houston, TX (US); Thomas Ray O'Brien, Houston, TX (US)

(21) Appl. No.: 14/150,551

(22) Filed: Jan. 8, 2014

(51) Int. Cl. A61K 8/36; A61K 8/86; A61K 8/04; A61K 8/34; A61K 8/97; A61K 8/21; A61Q 11/00; A61K 8/22

(52) U.S. Cl. CPC A61K 8/36; A61Q 11/00; A61K 8/86; A61K 8/22; A61K 8/345; A61K 8/97; A61K 8/21; A61K 8/042; A61K 2800/48; A61K 2800/592

(57) ABSTRACT

An dual oral composition is provided having a first peroxide-containing component and a second bicarbonate-containing component having exceptional anti-plaque and anti-bacterial action with re-mineralization properties.

### BACKGROUND OF THE INVENTION

1. Field of the Invention

The present invention relates to a dual composition dentifrice, more particularly to a composition of dentifrice that achieves significant effects for preventing dental caries and periodontal diseases.

2. The Related Art

Tooth decay is an infectious disease, the key feature of which is an increase within dental plaque of bacteria such as Streptococcus mutans and Lactobacillus. Tooth decay is an irreversible infection and causes demineralization of hard tissues. If left untreated the disease causes pain and infection.

De-mineralization produces organic acids when carbohydrates, especially sugar are eaten. When enough acid is produced so that PH goes below 5.5, this causes a process called de-mineralization. Teeth are porous allowing fluids and de-mineralization beneath the surface of the tooth enamel including the root below the gum line. When demineralization occurs, these pores become larger forming cavities. Re-mineralization is the process in which minerals are returned to the molecular structure of the tooth itself The present invention provides for a new composition that enhances the re-mineralization process.

### SUMMARY

An oral composition is provided including a bicarbonate and peroxide component. Accordingly, it is an object of the present invention to provide a dual component composition that enhances the re-mineralization process of tooth enamel wherein tooth decay and periodontal diseases are prevented. These and other objects of the present invention will become more readily apparent through consideration of the following summary and detailed description.

### DETAILED DESCRIPTIONS

The present invention comprises a dual component oral composition for the prevention of tooth decay. The dual composition comprises a first cleansing agent base gel and a second sodium bicarbonate paste. The dual composition is used in conjunction with one another in a 1:1 ratio. The cleansing agent of the first component can be hydrogen peroxide or potassium sorbate. Beyond the first essential ingredient of hydrogen peroxide or potassium sorbate of the first component there will also be included pharmaceutical acceptable carries including surfactant, humectants, thickening agent and sweeteners.

### Peroxide Gel Cleansing Agent

A first essential component or cleaning agent of compositions according to the present invention is that of a peroxide compound by way of hydrogen peroxide. The preferably amount of the peroxide compound is 15% by weight.

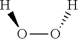

Polysorbate 20 NF is the surfactant which can be incorporated to provide foaming properties. Sorbitol Solution USP 70% by weight is the humectants or wetting agent which prevents the water in the composition from evaporating.

Sweetness and flavoring agents can be used to ensure good taste during tooth brushing or for a freshening aftertaste after tooth brushing. In this gel component, Saccharin Sodium USP Dihydrate is the sweetener and Methyl Salicylate NF and Peppermint Oil are the flavorings. Poloxamer 407 25% gel is the thickening agent. Hydrogen peroxide is the preferred essential cleaning agent which ranges between 7-15% by weight.

### Potassium Sorbate Cleansing Agent

In this alternative gel composition, Poloxamer 407 gel (25 ML) is the thickening agent and potassium sorbate of (0.3 GM) is the essential cleaning agent. Water is the humectants used as the wetting agent.

### Method of Preparation of Hydrogen Peroxide Gel

In an appropriate container mix saccharin sodium and polysorbate 20 in sorbitol solution until completely dissolved. Then add flavoring to the above mixture. Add hydrogen peroxide to the mixture and place in an ice bath until uniform mixture is form. Transfer mixture to containers and let stand at room temperature.

### Method of Preparation of the Potassium Sorbate Gel

Mix Potassium Sorbate and Poloxamer 407 25% gel in a container and bring to final volume with cold refrigerated purified water. Refrigerate resultant mixture when all granules of potassium sorbate are mixed thoroughly and it make take up to 12 to 24 hours within the refrigerator before the mixture becomes a clear solution. Once the mixture becomes a clear solution, the gel will solidify at room temperature.

### Second Component

The second oral composition comprises the following essential compounds: Sodium Bicarbonate, Xylitol, and a re-mineralization agent including Calcium Carbonate or Calcium Phosphate of, Dibasic USP Anhydrous of equivalent percentage of weight. Along with these compounds, the composition will also include sodium chloride (salt), flavorings, and a thickening agent. Polysorbate 20 NF is the thickening agent and the flavorings includes Spearmint oil or peppermint oil, citric acid USP monohydrate powder, marshmallow, stevioside 15% liquid extract, and spearmint powder. Sweetner is a Xylitol in this component.

A second essential component of compositions is that of sodium bicarbonate with a preferred weight of 25%.

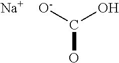

A third essential component of compositions according to the present invention is xylitol which is 0.1% by weight. Xylitol is a nonfermentable sugar alcohol which has dental health benefits in caries prevention. It has a plaque-reducing effect that attracts and starves harmful micro-organisms, allowing the mouth to re-mineralize damaged teeth with less interruption. Xylitol specifically inhibits the mutans streptococci group. It inhibits mutans streptococci in the presence of other sugars with the exception of fructose. Saliva containing xylitol is more alkaline than saliva which contains other sugar products. When saliva is alkaline, the calcium and phosphate salts in saliva start to precipitate into those parts of enamel where they are lacking thereby allowing the tooth to effectively re-mineralize.

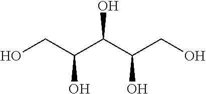

A fourth essential component of compositions is sodium fluoride that is 1.1% by weight. Amounts of the solution are optimally 4.5% by weight. This is a re-mineralization agent.

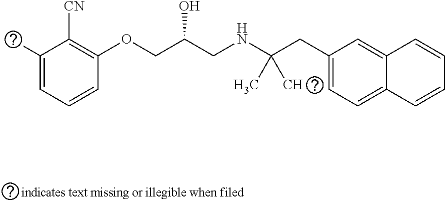 [SMILES: Cc1cccc(OC[C@H](O)CNC(C)(C)Cc2ccc3ccccc3c2)c1C#N]

A fifth essential compound of the present invention is calcium carbonate which is preferably 25% by weight

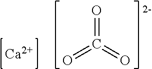

This is a re-mineralization agent. An alternative re-mineralization agent is calcium phosphate, Dibasic USP Anhydrous.

TABLE 1
Hydrogen Peroxide 150 MG/ML Oral Gel

| Ingredient | Weight | |
|---|---|---|
| Hydrogen Peroxide | 15 | ML |
| Saccharin Sodium | 0.03 | GM |
| Polysorbate 20 | 0.06 | ML |
| Sorbitol Solution 70% | 4.5 | ML |
| Methyl Salicylate | 0.3 | GM |
| Poloxamer 407 25% | 30 | ML |
| Peppermint Oil | .015 | ML |

The oral composition of the present invention for the hydrogen peroxide gel can be prepared in the preferred embodiment as described below in Table 1. Although Sorbitol is used below, Xylitol can be use as an alternative embodiment below.

The oral composition of the present invention for the sodium bicarbonate toothpaste can be prepared in the preferred embodiment as described below in Table 2.

TABLE 2

| | |
|---|---|
| Ingredient | Weight |

| | | |
|---|---|---|
| Sodium Fluoride | 1.1 | GM |
| Sodium Bicarbonate | 25 | GMS |
| Calcium Carbonate | 25 | GM |
| Sodium Chloride | 3 | GM |
| Polysorbate 20 | 27.8 | ML |
| Xylitol | .1 | GM |
| Citric Acid USP | 0.2 | GM |
| Monohydrate Powder | | |
| Flavor, Marshmallow, | 1.5 | ML |
| Artificial | | |
| Stevioside 15% Liquid | 3.5 | ML |
| Extract | | |
| Spearmint Powder | 2 | GM |

Sodium Fluoride/Sodium Bicarbonate Toothpaste

### Method of Preparation of Sodium Bicarbonate Paste

At room temperature in a mortar and pestle triturate Sodium Bicarbonate, Sodium Chloride and Calcium Carbonate together. Then wet with enough glycerin until a thick smooth paste is done. Then add flavorings and levigate thoroughly.

What is claimed is:

1. A dual component oral composition dentifrice for cleaning teeth comprising:
a gel base comprising:
a cleansing agent compound present in an amount of approximately 15% by weight of the gel base;
a humectant sugar alcohol component in an amount of approximately 4.5% by weight of the gel base; and
a paste compound comprising:
sodium bicarbonate in an amount of approximately 25% by weight of the paste component;
xylitol an anti-plaque forming agent with in an amount from 0.1% by weight of the paste component; and
and a-re-mineralization agent with a mineral composition that works in combination with the anti-plaque forming agent to re-mineralize damaged teeth.

2. The oral composition of claim 1 wherein the re-mineralization agent is from the group consisting of: calcium carbonate and calcium phosphate.

3. The oral composition of claim 1 wherein the gel base further comprises polysorbate 20 with an amount 6% by weight of the gel base.

4. The oral composition of claim 1 wherein the gel base further comprises sweeteners.

5. The oral composition of claim 1 wherein the gel base further comprises flavoring.

6. The oral composition of claim 1 wherein the cleansing agent is hydrogen peroxide.

7. The oral composition of claim 1 wherein the humectant is from the group consisting of Sorbitol and Xylitol.

8. A dual component oral composition dentifrice for cleaning teeth base comprising:
a potassium sorbate compound present in an amount of approximately 0.3% by weight of the gel base;
a humectant sugar alcohol component in an amount of approximately 4.5% by weight of the gel base; and
a paste compound comprising:
sodium bicarbonate in an amount of approximately 25% by weight of the paste component;
xylitol an anti-plaque forming agent with in an amount of approximately 0.1% by weight of the paste component; and
and a re-mineralization agent having a mineral composition that in combination with the anti-plaque forming agent to re-mineralize damaged teeth.

9. The oral composition of claim 8 wherein the re-mineralization agent is from the group consisting of:
calcium carbonate and calcium phosphate.

10. The oral composition of claim 8 wherein the gel base further comprises Poloxamer 407 with an amount 25% by weight of the gel base.

11. The oral composition of claim 8 wherein the gel base further comprises sweeteners.

12. The oral composition of claim 8 wherein the gel base/further comprises flavoring.

13. The oral composition of claim 8 wherein the humectant is from the group consisting of Sorbitol and Xylitol.
